(19) United States
(12) Patent Application Publication
(10) Pub. No.: US 2025/0050130 A1
(43) Pub. Date: Feb. 13, 2025

(54) LIGHT BASED THERAPY DEVICES AND METHODS

(71) Applicant: HAIR GROUP, LLC, Walnet Creek, CA (US)

(72) Inventors: Sara WASSERBAUER, Walnut Creek, CA (US); Erik CARLSON, Walnut Creek, CA (US); Ashwin GANESHAN, Walnut Creek, CA (US); Guoqiang LI, Walnut Creek, CA (US)

(21) Appl. No.: 18/931,032

(22) Filed: Oct. 29, 2024

Related U.S. Application Data
(63) Continuation-in-part of application No. 17/697,913, filed on Mar. 17, 2022.
Application No. 17/697,913 is a continuation-in-part of application No. 16/989,607, filed on Aug. 10, 2020, now abandoned.
Application No. 16/989,607 is a continuation of application No. 16/681,109, filed on Nov. 12, 2019, now Pat. No. 10,773,097.
Application No. 16/681,109 is a continuation of application No. 15/677,840, filed on Aug. 15, 2017, now Pat. No. 10,525,278.

(51) Int. Cl. A61N 5/06

(52) U.S. Cl. CPC A61N 5/0616; A61N 2005/063; A61N 2005/0647; A61N 2005/0651

(57) ABSTRACT

The present invention relates to a light therapy apparatus, system, and method to deliver light to a body organ, e.g., to directly contact the skin such as a scalp, via one or more light guides in therapeutic dosages from one or more light sources operating in a variety of wavelengths. One or more illumination assemblies may be fixedly coupled to two or more panel assemblies operably coupled to a substructure. The panel assemblies may be acted upon by one or more actuators to move the panel assemblies resulting in conformal contact with the body organ from a first position to a second position. The actuator may comprise a cable coupled to a motor, the cable operably coupled to each panel assembly, so that upon activation of the motor, the panel assemblies each move inwardly in an independent direction with respect to each other to conform to the body organ.

### RELATED APPLICATIONS

This application is a continuation-in-part of co-pending U.S. patent application Ser. No. 17/697,913 filed on Mar. 17, 2022, entitled “Light Based Therapy Devices and Methods”, which is a continuation-in-part of Ser. No. 16/989,607 filed Aug. 10, 2020, entitled “Light Based Therapy Devices and Methods”, which is a continuation of U.S. patent application Ser. No. 16/681,109 filed Nov. 12, 2019, entitled “Light Based Therapy Devices and Methods”, now U.S. Pat. No. 10,773,097 issued Sep. 15, 2020, which is a continuation of U.S. patent application Ser. No. 15/677,840 filed Aug. 15, 2017, entitled “Light Based Therapy Devices and Methods”, now U.S. Pat. No. 10,525,278 issued Jan. 7, 2020. Each of these references are incorporated herein by reference in their entireties.

### BACKGROUND

The present invention relates to designs, systems, and methods of a light therapy device.

One major application of Low Level Light Therapy (LLLT) is to treat hair with photobiomodulation in the wavelengths of 614-624 nm, 668-684 nm, 751-772 nm, and 813-846 nm that has been proven to reduce inflammation in the scalp, stimulate the release of growth factors in the hair follicle, up-regulate the production of ATP, that is the energy source for the cell, and increase oxygen levels and blood flow via a vasodilatory effect. Devices of all sorts include caps, combs, helmets, handheld “massager-type” units, and hoods. These conventional devices have problems regarding hair absorption of the light intended for the skin and the reflective properties of the skin. Therapeutic dosing also is difficult as these two effects affect precise control thereof during therapeutic applications since the applied LLLT light can be scattered, absorbed, transmitted, or reflected. Consequently, there is a need for a LLLT device to overcome absorption and other problems to improve therapeutic dosing of LLLT transmitted light applied to the surface for consistent dosing thereof.

### SUMMARY

In one aspect, the present disclosure provides variations of improved systems, methods, and devices for providing LLLT devices. Such devices are suited for hair growth by applying light delivery to the skin using one or more illumination sources. The present invention relates to a device for positioning over and applying electromagnetic energy to a body organ, the device comprising a dome forming a contoured shape, said dome formed by an array of panel assemblies, where an interior space of the contoured shape is adapted for placement over the body organ. The device may include an arrangement where each panel assembly comprises a plurality of illumination assemblies rigidly fixed with respect to each other and the panel assembly to which it is attached, and where each illumination assembly includes an optical assembly having a proximal end optically coupled to a light source, a distal end configured to direct electromagnetic energy to the body organ, and one or more light guides therebetween, each of said illumination assemblies being coupled to the dome to allow the optical assembly to extend past and within the dome towards the body organ. Each panel assembly may be configured to bias toward or away from the body organ, in a quasi-independent manner, in accordance with the size and shape of that body organ of a particular individual. Each panel assembly of the array may move in one or more directions that are independent direction(s) moved by the other panel assemblies; this movement may be spherical in nature whereby the panels move globally inward or outward to expand or contract the sphere, which is generally represented by the contoured shape of the dome. The device may use an actuator, such as a motor, configured to wind or unwind a circumferential cable threaded through each panel assembly, whereupon activation, the actuator moves the array from a first position to a second position corresponding to a conformal arrangement around a body organ, and upon deactivation, to move the said plurality of panel assemblies back to said first position.

Variations of the device and system include illumination sources that are shaped for patient comfort and/or to distribute the light around the delivery or contact point. In additional variations, a projecting element that includes or carries the illumination source is actuated so as to allow conformal contact with the skin. An active system to cause conformal contact with the skin may include one or more actuation types, including but not limited to hydraulic, pneumatic, electrical, thermal, magnetic, and/or soft actuators. In an exemplary embodiment, an array of such projection elements having illumination sources are used to illuminate a substantial area of skin, such as the scalp. An advanced passive cooling scheme may be used to preserve the output power and efficiency of the light sources. Advantageously, the present invention delivers light directly to the skin, bypassing interference from hair shafts, thereby allowing for a known dosing intensity and distribution. The configurations described herein can provide an improvement in light delivery to the targeted region-one that reduces loss of energy to undesired absorption and reflection and that ensures maximum absorption by the target tissue, thereby enabling standardized dosing. Variations of the devices described herein can also allow delivery of light at or very close to the skin/scalp, which allows bypassing the interference that even short hair shafts above the skin create, and minimizing the effect of reflection. Additionally, the present invention may deliver light in one or more of the four optimum wavelength ranges.

Variations of the device also allow for a hands-free, cordless, and portable embodiments, with an interactive feedback component that allows a patient to monitor their progress, thereby further improving adherence with the treatment regimen. Such variations also time the treatments and help patients manage dosing frequency with a minimal amount of external visibility. Cell proliferation (i.e., growth of hair) is optimized with low doses over longer periods of time. So, the present LLLT device allows for convenient and frequent dosing (at least 2-3 times per week, if not daily).

Variations of the device additionally provide for ease of manufacturing, assembly, serviceability, and/or replacement of parts throughout the useful lifetime of the device. Such desirable effects may be achieved through minimization of parts, simplification of parts, and other novel approaches as described herein.

Other desirable features and characteristics will become apparent from the subsequent detailed description, drawings, and appended claims and/or abstract, when considered in view of this background and summary.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, which are incorporated in and constitute a part of this specification, show certain aspects of the subject matter disclosed herein, and, together with the description, help explain some of the principles associated with the disclosed implementations, wherein:

FIG. 1 illustrates a perspective view of a light therapy device for the scalp, including a plurality of illumination assemblies and an outer shell, according to an embodiment of the present invention;

FIG. 2 illustrates a perspective view of a light therapy device for the scalp, including a substructure, according to an embodiment of the present invention;

FIG. 3 illustrates a perspective view of a light therapy device for the scalp, including a plurality of biasing assemblies, wherein each biasing assembly is operably coupled to a panel assembly, according to an embodiment of the present invention;

FIGS. 4 illustrates a perspective view of exemplary LED strings configured with flex ribbon, according to an embodiment of the present invention;

FIG. 5 illustrates a perspective view of a light therapy device for the scalp, including a plurality of panel assemblies, according to an embodiment of the present invention;

FIG. 6 illustrates a cross-sectional view of a light therapy device for the scalp, taken along the line shown in FIG. 1, the light therapy device including a biasing assembly, according to an embodiment of the present invention;

FIG. 7 illustrates a top-down view of a light therapy device illustrating an exemplary plurality of panel assemblies, according to an embodiment of the present invention;

FIG. 8 illustrates a top-down view of a light therapy device illustrating an exemplary plurality of panel assemblies and corresponding biasing assemblies, according to an embodiment of the present invention;

FIG. 9A is a schematic view of a first position of a light therapy device illustrating including a plurality of panel assemblies and a cable coupled thereto, according to an embodiment of the present invention;

FIG. 9B is a schematic view of a second position of a light therapy device illustrating including a plurality of panel assemblies and a cable coupled thereto, according to an embodiment of the present invention; and

FIGS. 10A-10E illustrate views of an illumination assembly including a light source and an optical assembly, according to an embodiment of the present invention.

### DETAILED DESCRIPTION

Non-limiting embodiments of the invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention. The drawings featured in the figures are provided for the purposes of illustrating some embodiments of the invention and are not to be considered as limitation thereto.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

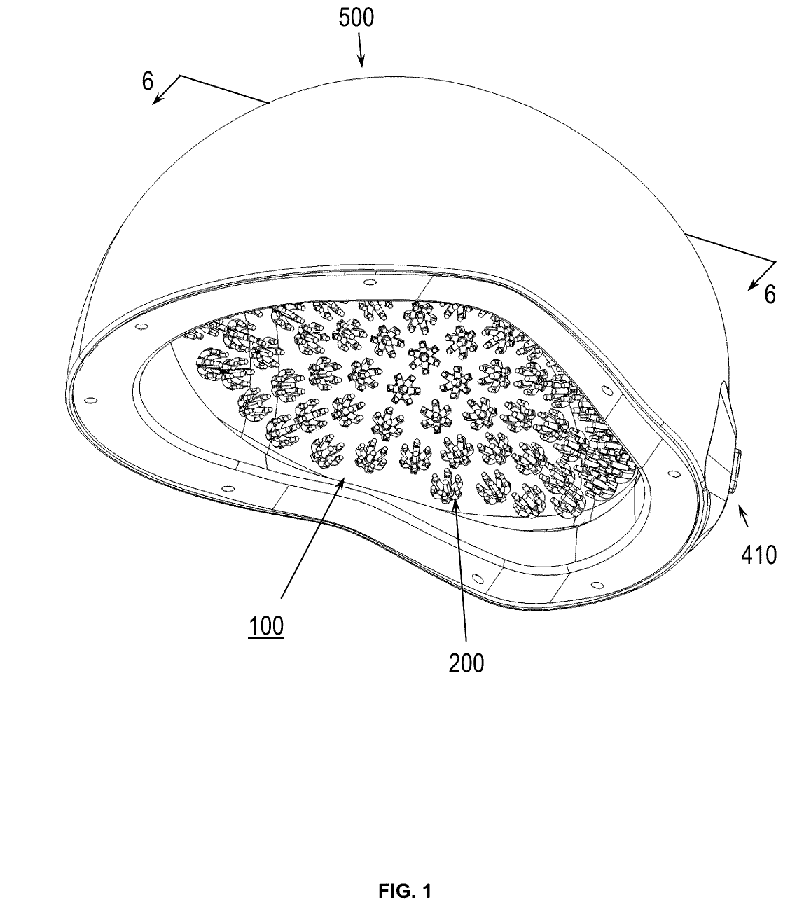

FIG. 1 provides a perspective view of an exemplary light therapy device, assembly, system and method for therapeutic treatment generally identified as element 100. The light therapy device 100 is described in an exemplary embodiment for therapeutic LLLT light therapy treatment of a scalp of a patient illustrating the concepts and features in the environment of the light therapy device worn on the head of the patient as shown, for example, as illustrated in FIG. 15 of U.S. patent application Ser. No. 17/697,913, which is commonly owned by the Applicant, although the particular features, structures, or characteristics may be combined in any suitable manner in one or more embodiments without limitation. Other uses for therapeutic LLLT light therapy treatment can comprise the device 100 configured for an entirety of a body structure (such as a hand, leg, head, scalp, etc.) for treatment of the skin or tissue that is part of such a body structure.

Referring to FIGS. 1-10E, the light therapy device 100 may comprise a substructure 120 rigidly coupled within an outer shell 500, one or more biasing assemblies 130 operably coupled to the substructure 120, and one or more panel assemblies 140, wherein each panel assembly 140 may be operably coupled to a corresponding biasing assembly 130. Outer shell 500 may comprise one or more openings 410. Openings 410 may be useful for venting of the light therapy device 100, such as for mechanical cooling, or may be useful for electrically charging ports, etc., so as to facilitate operation of the light therapy device 100.

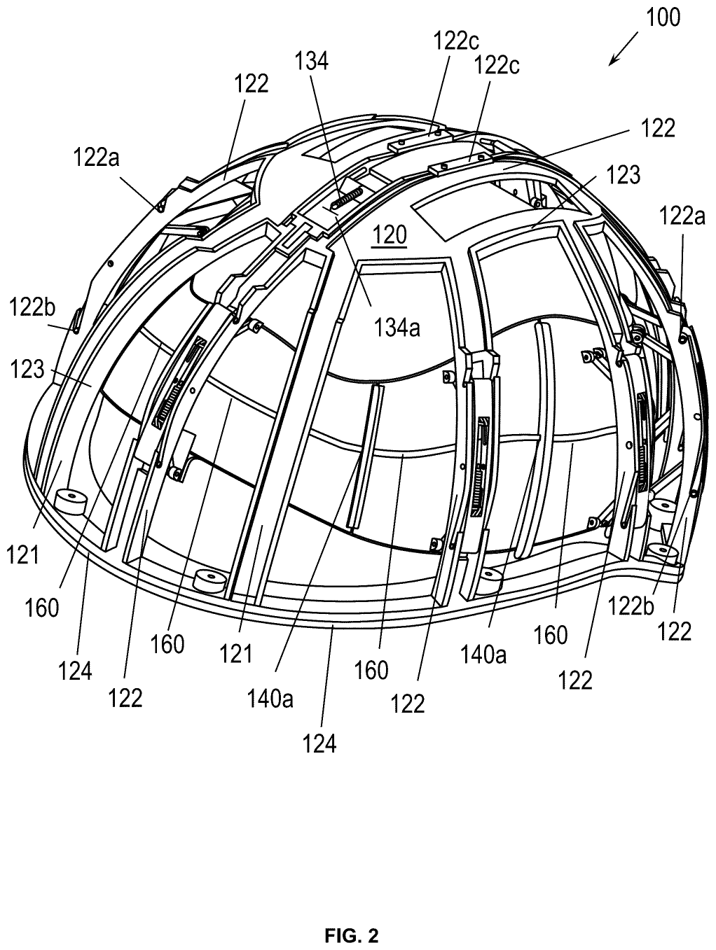

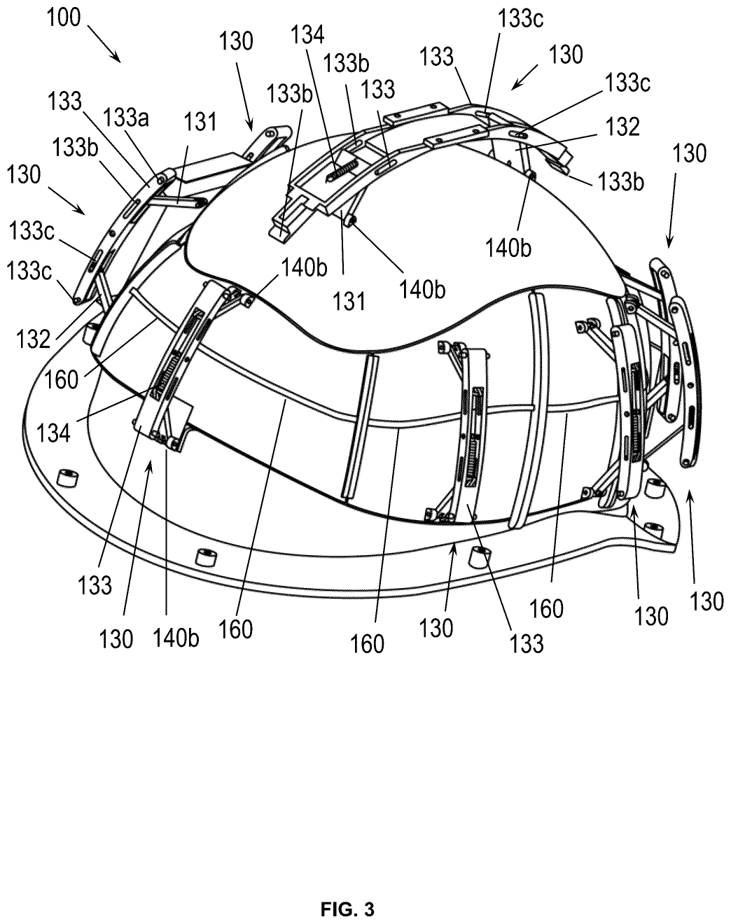

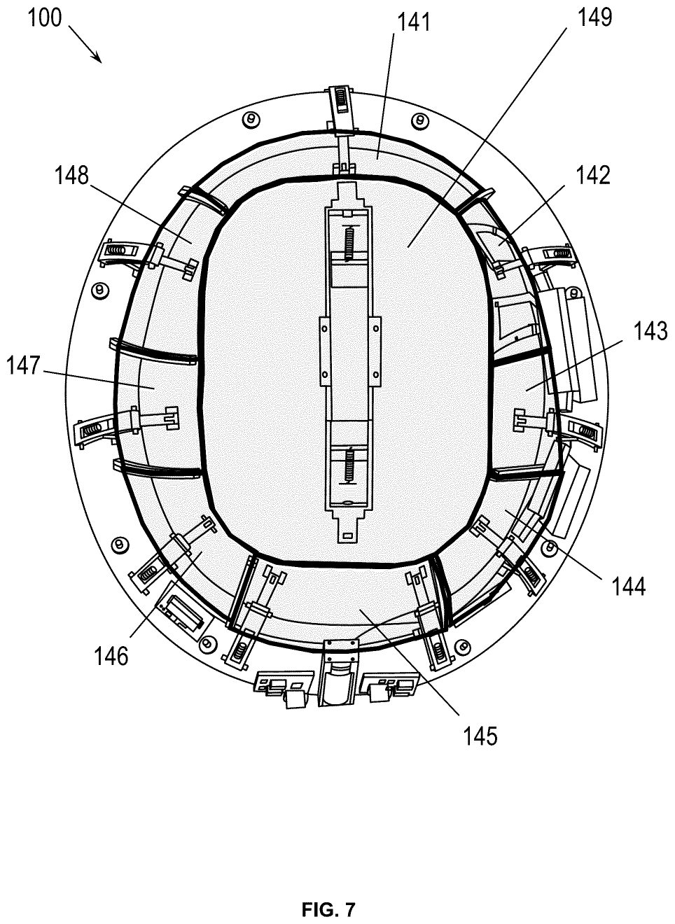

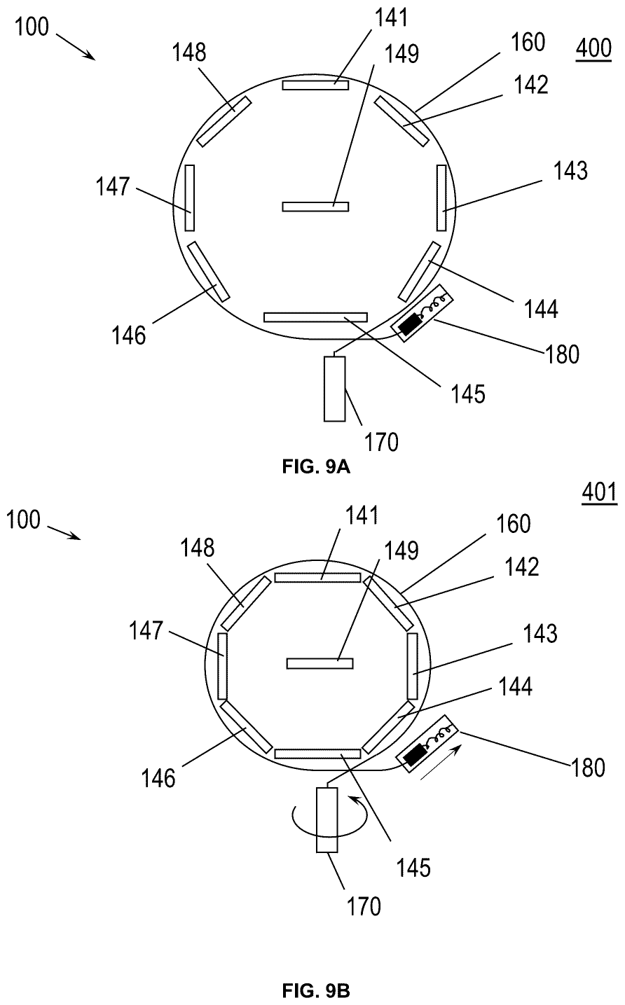

Referring to FIGS. 2-3, the light therapy device 100 may further comprise one or more illumination assemblies 200 that may be rigidly coupled to a corresponding panel assembly 140. In an assembled configuration, a cable 160 may be threaded through two or more panel assemblies 140 through a corresponding panel cable 160 opening 140a, so that the cable 160 operable couples the two or more panel assemblies 140, and the cable may be coupled to an actuator 170 at one end, and optionally a biasing compensator 190 at the other end. In operation, winding the cable 160 around a spindle proximate the actuator 170 causes the cable 160 to tighten, thereby circumferentially contracting the panel assemblies 140 inwardly so as to tighten about the scalp. One or more biasing elements 134 may be employed to bias the panel assemblies 140 in a first, or open, position 400 as in FIG. 9A, and upon actuation of actuator 170, a counter-balancing force causes the panel assemblies 140 to move to a second, or closed, position 400 as in FIG. 9B. A panel assembly 140 may be disposed at the top, i.e., the upper-most portion of the light therapy device 100, in a biased closed position, which may be an opposite resting position as compared to the circumferential panel assemblies. The upper-most panel assembly may be a ninth panel assembly 149 as shown in FIGS. 7-8 and 9A-9B. When the user puts on the light therapy device 100, it may be configured so that the scalp first touches this upper-most panel assembly 140, 149, and the weight of the light therapy device 100 causes the panel assembly 140, 149 to move upward relative to the outer shell thereby counter-balancing the biasing of that panel 140, 149 against the weight of the device 100. Subsequently, the user may activate the actuator 170 to thereby bring the circumferential panel assemblies 140 into an appropriate, predetermined contact with the scalp. This achieves contact of the panel assemblies 140 in a manner that adequately conforms to the unique characteristics and shape of the particular user's scalp.

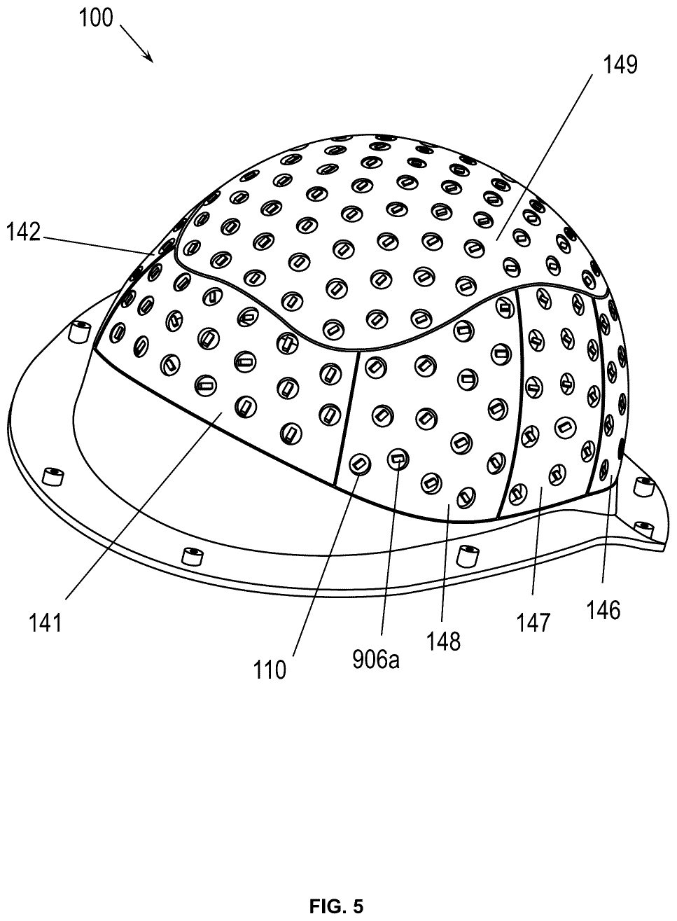

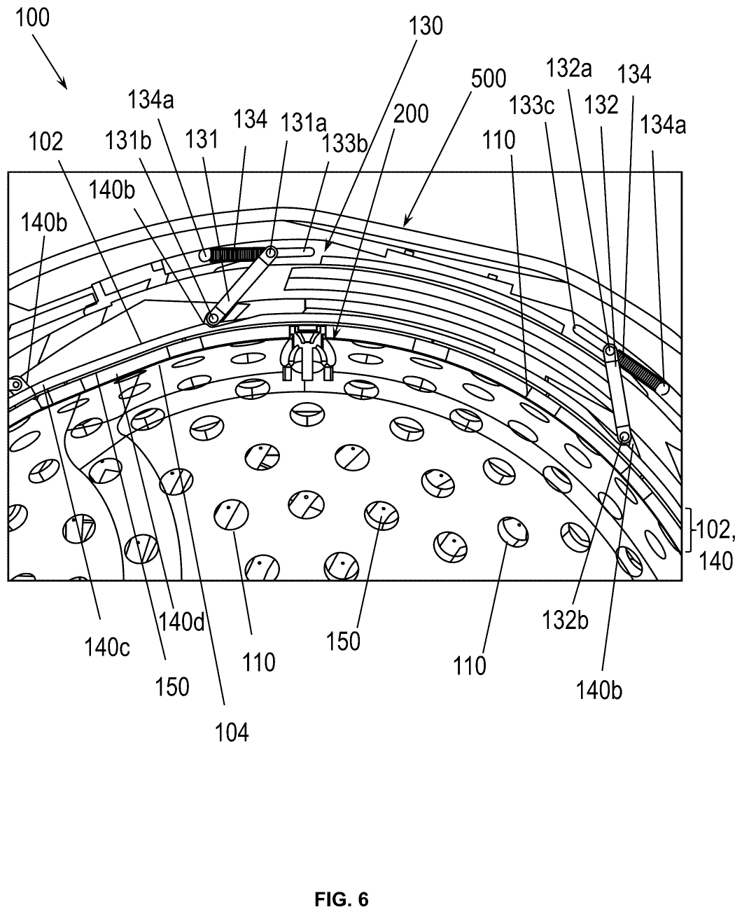

A plurality of panel assemblies 140 may cumulatively form a dome 102 shape, as shown in FIG. 6, wherein each panel assembly may be configured with a plurality of openings 110, as shown in FIG. 5, for a plurality of illumination assemblies 200. In FIG. 6 only one illumination assembly 200 is shown for the purpose of clarity. Generally, each illumination assembly 200 may extend into the cavity of the dome 102 through an exterior surface 106 of the dome 102 past the interior surface 104, so that at least a portion of each illumination assembly extends inwardly from interior surface 104 so that the illumination assembly 200 may touch, or be disposed proximate, the body organ. Each illumination assembly 200 may be configured to couple to the dome 102 via a press fit, or through the introduction of an adhesive, or other coupling method, so as to facilitate manufacture, testing, assembly, repair, operation and/or use thereof. As noted herein, the illumination source 200 is adapted to transmit light to the tissue of the scalp from a source of illumination.

Referring to FIG. 2, the light therapy device 100 is shown separately with an outer shell 500 removed. The light therapy device 100 may comprise a substructure 120 that may include a bottom loop 124 configured to couple to the outer shell 500. The bottom loop may couple to one or more supports 121 and one or more biasing assembly supports 122. Each biasing assembly support 122 may include an upper receiver portion 122a and a lower receiver portion 122b, which may be configured to couple to a biasing assembly 130 as detailed in FIG. 3. The substructure 120 may be generally characterized as including one or more ribs 123 that form at least a portion of the bottom loop 124, the supports 121 and/or the biasing assembly support 122, where the ribs 123 provide structural support of the substructure 120, while reducing the overall weight of the light therapy device 100. A top receiver portion 122c may be disposed proximate the top of the substructure 120, for coupling to a biasing assembly 130.

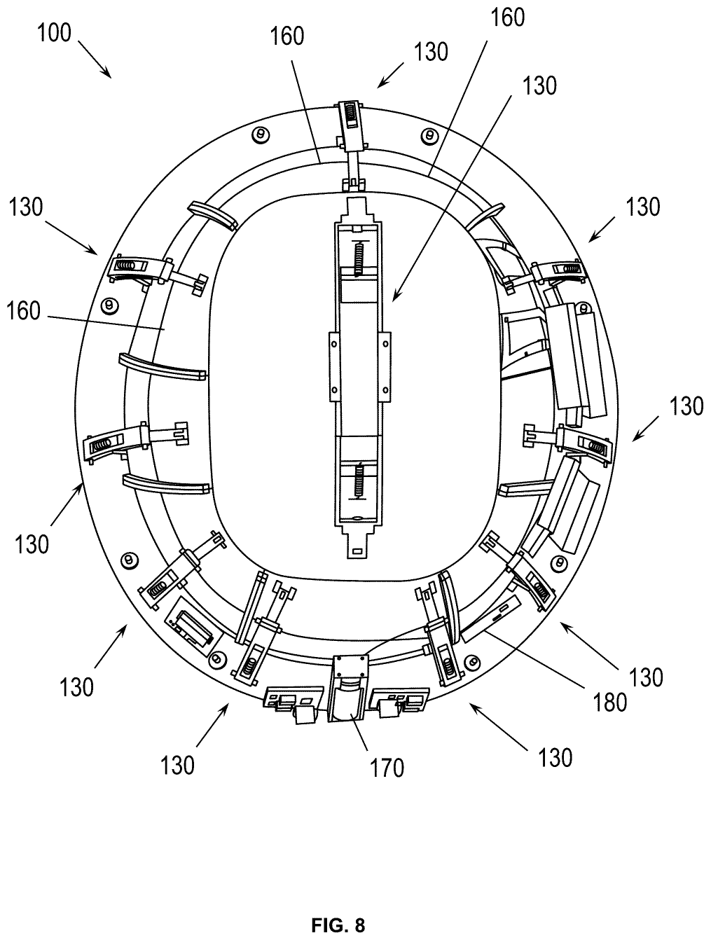

Referring to FIG. 3, the light therapy device 100 is shown with the outer shell 500 and the substructure 120 removed. The light therapy device 100 may comprise one or more biasing assemblies 130. In one embodiment, the biasing assemblies 130 may be arrayed circumferentially around the dome 102, as shown in FIG. 8. The light therapy device 100 may also include a biasing assembly 130 disposed proximate the top thereof, also shown in FIG. 8. Each biasing assembly 130 may comprise an upper biasing arm 131, a lower biasing arm 132, a slider body 133, and a biasing element 134.

Certain features of the biasing assembly 130 can be viewed in FIG. 6 with reference to FIG. 3, and/or with reference to FIGS. 2 and 7-8. Each upper biasing arm 131 may include an upper biasing arm slot pin 131a and an upper biasing arm panel pin 131b. Each lower biasing arm 132 may include a lower biasing arm slot pin 132a and a lower biasing arm panel pin 132b. Each slider body 133 may include a slider body upper pin 133a, a slider body upper slot 133b, a slider body lower slot 133c, and a slider body lower pin 133d. The slider body 133 may couple to the substructure 122 via the slider body upper pin 133a coupling to the upper receiver portion 122a, and via the slider body lower pin 133d coupling to the lower receiver portion 122b. This may fixedly couple the slider bodies 133 to the substructure 122, and to the outer shell 500. The slider body 133 may couple to the upper biasing arm 131 via operable coupling of the slider body upper slot 133b to the upper biasing arm slot pin 131a. The slider body 133 may couple to the lower biasing arm 132 via operable coupling of the slider body lower slot 133c to the lower biasing arm slot pin 132a. In this way, each of the upper and lower biasing arms 131, 132 may rotate and/or translate relative to the slider body 133.

Referring to FIGS. 7 and 8, one or more panel assemblies 140 may operably couple to a corresponding biasing assembly 130. In one embodiment, as in FIG. 7, the light therapy device 100 may comprise a first panel assembly 141, a second panel assembly 142, a third panel assembly 143, a fourth panel assembly 144, a fifth panel assembly 145, a sixth panel assembly 146, a seventh panel assembly 147, an eighth panel assembly 148, and a ninth panel assembly 149. This arrangement is exemplary, and the light therapy device 100 is capable of any other quantity and arrangement of panel assemblies 140 arrayed to form a dome 102, and therefore the invention is non-limiting with at least respect to this regard.

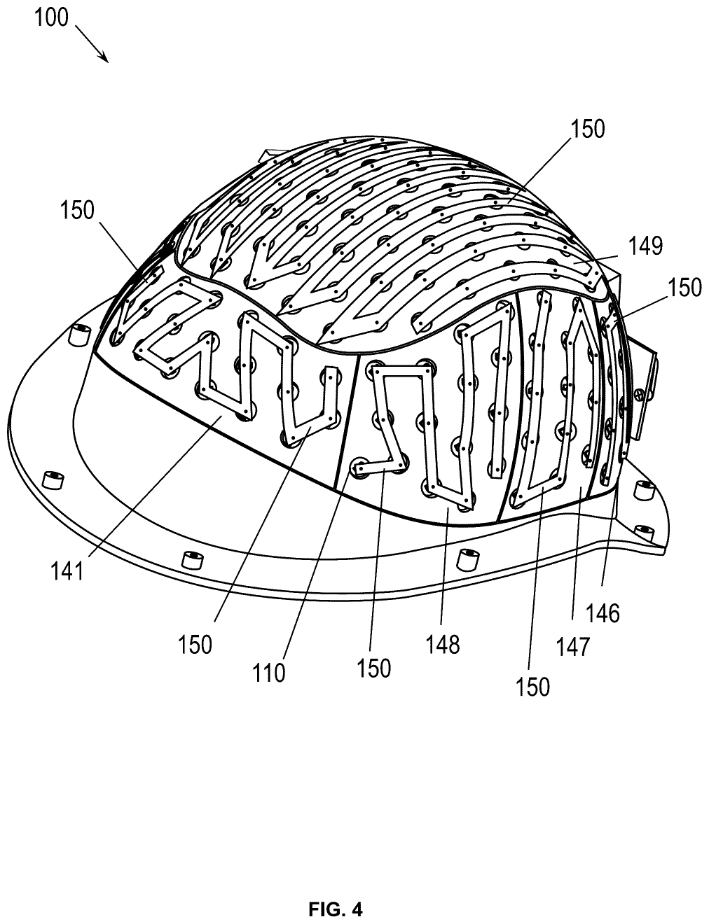

Each panel assembly 140, such as exemplary first through ninth panel assemblies 141-149, may comprise a panel cable opening 140a, a panel pin holder 140b, an outer panel layer 140c, and an inner panel layer 140d, as illustrated in FIGS. 2-8. In alternative embodiments, each of the outer and inner panel layers 140c and 140d may comprise multiple layers of material; alternatively, these may effectively constitute a single layer, and the invention 100 is non-limited thereby. The outer and inner panel layers 140c and 140d may shield and protect electrically materials and connections, such as flex ribbons 150 as shown in FIG. 4. Referring to FIGS. 3 and 6, each panel assembly 140 may be configured to operably couple to a corresponding biasing assembly 130. For example, a plurality of panel pin holders 140b may be fixedly coupled to outer panel layer 140c. As visible in FIG. 6, a panel pin holder 140b may couple to an upper biasing arm panel pin 131b, and a panel pin holder 140b may couple to a lower biasing arm panel pin 132b; each coupling point may operate as a pin connection to pivotably adjust the panel assembly 140 relative to the slider body 133.

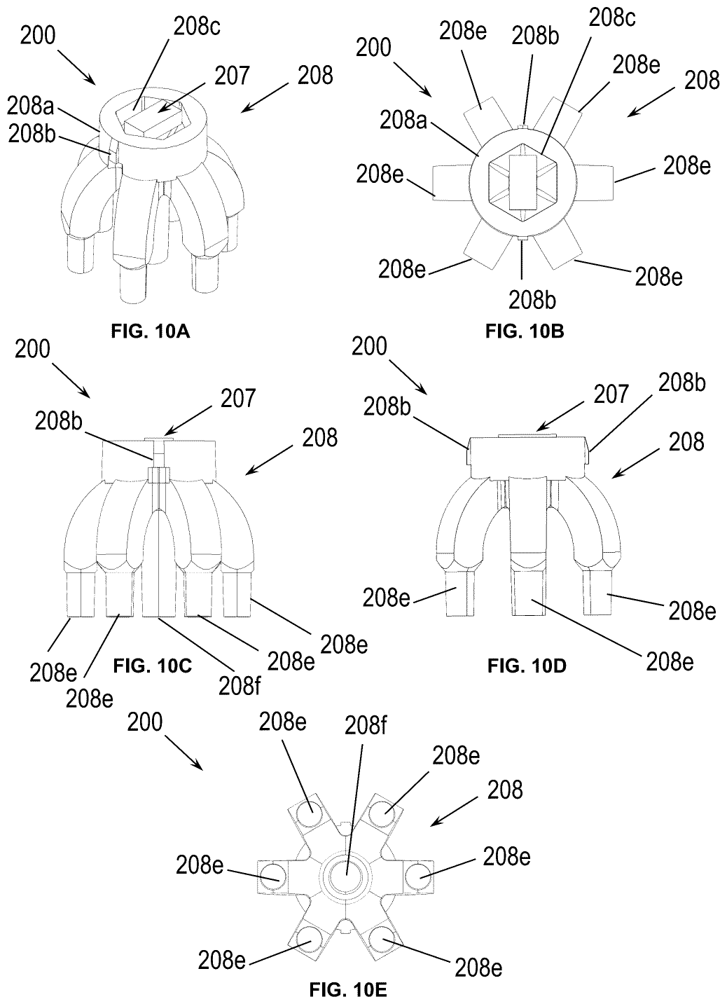

Referring to FIGS. 10A-10E, an illumination assembly 200 is illustrated. Illumination assembly 200 may comprise a light source 207 coupled to an optical assembly 208. Optical assembly 208 may comprise base 208a. One or more tabs 208b may extend outwardly from said base 208a to facilitate the fixed coupling of the illumination assembly 200 within an opening 110 of inner panel layer 140d of panel assembly 140. Optical assembly 208 may further comprise a lens 208c for directing light from the light source 207 to light guides, where the light guides direct the light to the scalp. In a preferred embodiment, to facilitate the equal distribution of light across the surface area of the scalp, optical assembly 208 may further comprise one or more auxiliary light guides 208e radially-arrayed around a central light guide 208f. The reader is directed to U.S. patent application Ser. No. 17/697,913, commonly owned by the Applicant, for further explanation as to the preferred characteristics of the light source 207 and other similar elements of the light therapy device 100.

In an assembled configuration, and in a preferred embodiment, each of the circumferential panel assemblies 140, such as first through eighth panel assemblies 141-148, may be biased open, as in a first position 400 shown in FIG. 9A. In the preferred embodiment the top panel assembly 140, such as ninth panel assembly 149 may be biased closed, which corresponding to the panel assembly 140 being disposed inwardly relative to the outer shell 500. When the user puts on the light therapy device 100, the top panel assembly 140, such as the ninth panel assembly 149 may couple to the scalp of the user, and bias to a second position, corresponding to the ninth panel assembly 149 moving outwardly toward the outer shell 500. The ninth panel assembly 149 may exhibit this moving characteristic as the weight of the light therapy device is transmitted through the ninth panel assembly 149 to rest upon the scalp. The exemplary biasing assembly 130 and panel assembly 140 shown in FIG. 6 corresponds to the top panel assembly 140, such as the ninth panel assembly 149, but has an arrangement. The ninth panel assembly 149 may be biased open by virtue of the location and arrangement of the parts shown in FIG. 6; in particular, the biasing element 134 of FIG. 6 may be coupled to a biasing element holder 134a (see also FIG. 2) in a manner so as to pull upper biasing arm slot pin 131a away from the center of the biasing assembly 130 and/or the center of the panel assembly 140, 149. This arrangement facilitates the ninth panel assembly 149 to bias closed in a resting position. In the preferred embodiment, and in contradistinction to ninth panel assembly 149, each of the circumferential panel assemblies 140, such as first through eighth panel assemblies 141-148, may be biased open. In panel assemblies 141-148, it may be preferrable to therefore dispose each biasing element 134 so that it moves pins 131a and 132a toward the center of the biasing assembly 130 and/or toward the center of the panel assembly 140, 141-148. This implies that the circumferential panel assembly 141-148 may be disposed outwardly toward outer shell 150, in the resting position, to thereby accommodate the user's placement of their scalp into the light therapy device 100. Each of the one or more biasing elements 134 used in conjunction with a biasing assembly 130 may be configured to couple to a biasing element holder 134a, which may in turn be coupled to a component that is fixedly attached relative to the outer shell 500; for example, the biasing element holder 134a may couple to the slider body 133, or it may couple to the support 121.

To operate the light therapy device 100, the user may then turn ON the actuator 170, such as a motor, to cause a cable 160 to tighten and move each of the circumferential panel assemblies 140, such as first through eighth panel assemblies 141-148, from a first position and/or biased open position, 400 as in FIG. 9A to a second position and/or biased closed position 401 as in FIG. 9B, having the light guides 208e in contact with the scalp to direct the light to the scalp. The therapy sequence may commence upon successful position of the light therapy device 100. Upon completion of the therapy sequence, the reverse operation may take place, such as the user turning OFF the actuator 170 causes the circumferential panel assemblies 140 to return to the first position such as, for example, first through eighth panel assemblies 141-148, to move from a biased closed position 401 to a biased open position 400.

Referring again to FIGS. 9A-9B, the light therapy device 100 may include biasing characteristics that counterbalance the system so that proper tensioning of the cable 160 is achieved in both open and closed positions 400, 401, respectively. For example, the biasing compensator 180 provides a countering force to the actuator 170, which results in a smoother operation at, and between, each position 400, 401. The actuator 170 may include a motor and a barrel/spindle that overcomes the tension of the biasing compensator 180 when turn ON. In a preferred embodiment, this arrangement of parts may result in a total diametral distance delta of 10 mm between the opened 400 and closed 401 positions. This arrangement results in the light therapy device 100, for example, accommodating heads of different sizes, while providing a holding/coupling force that is adequate without imparting too much force on the user to cause discomfort.

A plurality of illumination assemblies 200 may be fixedly coupled to each panel assembly 140 in a spatially arrayed manner to facilitate adequate coverage of light to the body organ, for example, the skin of the scalp. Each panel assembly 140 is in effect independently biased so that the illumination assemblies 200 can conform to the shape of the head and/or be operably in contact with the body organ. This independent biasing allows for an improved device with multiple illumination sources that achieves independent contact of each panel assembly 140, and/or each illumination assembly 200, to conform to a contoured surface of the body organ without significantly affecting adjacent assemblies, which increases the ability of the adjacent assemblies to irradiate the tissue, e.g., in a normal direction. Lambert's cosine law predicts that an illumination beam that is perpendicular to its target can achieve a higher rate of irradiance. In contrast, a beam that is offset from a perpendicular approach will have a greater degree of reflection off of the target area; so, an illumination beam will have the highest absorption for a curved or contoured body organ like the surface of the scalp, when that illumination irradiates to the scalp at a perpendicular angle. The independent biasing of the panel assemblies 140 across a localize region of the scalp, therefore, can decrease the overall reflectance of light from a curved surface and increase the effectiveness or uniformity of the applied LLLT light therapy. Consequently, the dome 102 advantageously maintains a normal orientation of each illumination assembly 200 as shown in FIG. 1.

The previous description of the disclosed embodiments is provided to enable any person skilled in the art to make or use the present invention. Various modifications to these embodiments will be readily apparent to those skilled in the art, and the generic principles defined herein can be applied to other embodiments without departing from the spirit or scope of the invention. For example, a wide variety of materials may be chosen for the various components of the embodiments. It is therefore desired that the present embodiments be considered in all respects as illustrative and not restrictive, reference being made to the appended claims as well as the foregoing descriptions to indicate the scope of the invention.

What is claimed is:

1. An apparatus for positioning over and applying electromagnetic energy to a body organ, the apparatus comprising:
a substructure including a bottom loop and two or more biasing assembly supports extending therefrom, each biasing element assembly support including an upper receiver portion and a lower receiver portion;
two or more biasing assemblies, each biasing assembly including:
a slider body having a slider body upper pin, a slider body upper slot, a slider body lower slot, and a slider body lower pin, wherein the slider body upper pin is adapted to couple to the upper receiver portion, and the slider body lower pin is adapted to couple to the lower receiver portion,
an upper biasing arm having an upper biasing arm slot pin and an upper biasing arm panel pin, wherein the upper biasing arm slot pin is adapted to couple to the slider body upper slot,
a lower biasing arm having a lower biasing arm slot pin and a lower biasing arm panel pin, wherein the lower biasing arm slot pin is adapted to couple to the slider body lower slot, and
a biasing element adapted to couple, on a first end, either to the upper biasing arm slot pin or to the lower biasing arm slot pin, the biasing element further adapted to couple, on a second end, to a biasing element holder formed either on the slider body or on the substructure;
two or more panel assemblies, each panel assembly including two or more panel pin holders, and a panel having a least one panel opening extending therethrough, wherein the upper biasing arm panel pin is adapted to couple to the first panel pin holder, and the lower biasing arm panel pin is adapted to couple to the second panel pin holder; and
a plurality of illumination assemblies, each illumination assembly including a light source and an optical assembly, wherein each illumination assembly is adapted to couple to one of the corresponding panel openings,
wherein, in an assembled configuration and upon application of an external force, each of the two or more panel assemblies is adapted to move between a first position and a second position, the external force counteracting the tension of the biasing element, each panel moving in an independent direction with respect to each other panel.

2. The apparatus of claim 1, wherein the panel further comprises an outer panel layer and an inner panel layer, and an electrical layer disposed therebetween, the at least one panel opening extending through the inner panel layer, and the light source adapted to couple to the electrical layer.

3. The apparatus of claim 1, further comprising an actuator adapted to exert the external force upon at least one of the two or more panel assemblies to move from the first to the second position, and wherein the biasing elements bias the two or more panel assemblies in an OPEN position corresponding to the first position, and where the second position corresponds to a CLOSED position.

4. The apparatus of claim 3, wherein the actuator comprises:
a motor including a winding spindle; and
a cable routed circumferentially within the apparatus and threaded through a panel cable opening formed on or within each panel of the two or more panel assemblies,
wherein the motor causes the external force to tighten the cable about the winding spindle and thereby move the two or more panel assemblies inwardly from the first position to the second position.

5. The apparatus of claim 4, wherein the actuator further comprises a biasing compensator for smoothing the movement of apparatus.

6. The apparatus of claim 3, further comprising a top panel assembly coupled to a top biasing assembly, wherein the one or more biasing elements of the top biasing assembly bias the panel of the top panel assembly in a CLOSED position corresponding to the first position, and where the second position corresponding to an OPEN position.

7. The apparatus of claim 3, wherein said actuator is selected from the group consisting of: a biasing actuator, a hydraulic actuator, a pneumatic actuator, an electrical actuator, a thermal actuator, a magnetic actuator, and a soft actuator.

8. The apparatus of claim 1, wherein the optical assembly comprises a base including a lens formed therein, one or more auxiliary light guides and a central light guide, each configured to direct light from the light source to the body organ.

9. The apparatus of claim 1, wherein the substructure further comprises one or more supports to add structural rigidity to the apparatus.

10. A helmet for administering light therapy to the scalp, the helmet comprising:
a substructure including a bottom loop and two or more biasing assembly supports extending therefrom, each biasing element assembly support including an upper receiver portion and a lower receiver portion;
two or more biasing assemblies, each biasing assembly including:
a slider body having a slider body upper pin, a slider body upper slot, a slider body lower slot, and a slider body lower pin, wherein the slider body upper pin is adapted to couple to the upper receiver portion, and the slider body lower pin is adapted to couple to the lower receiver portion,
an upper biasing arm having an upper biasing arm slot pin and an upper biasing arm panel pin, wherein the upper biasing arm slot pin is adapted to couple to the slider body upper slot,
a lower biasing arm having a lower biasing arm slot pin and a lower biasing arm panel pin, wherein the lower biasing arm slot pin is adapted to couple to the slider body lower slot, and
a biasing element adapted to couple, on a first end, either to the upper biasing arm slot pin or to the lower biasing arm slot pin, the biasing element further adapted to couple, on a second end, to a biasing element holder formed either on the slider body or on the substructure;
two or more panel assemblies, each panel assembly including a panel cable opening, two or more panel pin holders, and a panel having a least one panel opening extending therethrough, wherein the upper biasing arm panel pin is adapted to couple to the first panel pin holder, and the lower biasing arm panel pin is adapted to couple to the second panel pin holder;
a plurality of illumination assemblies, each illumination assembly including a light source and an optical assembly, wherein each illumination assembly is adapted to couple to one of the corresponding panel openings; and
an actuator comprising:
a motor, and
a cable including a first cable end, a second cable end, and a cable length disposed therebetween, wherein the first cable end is coupled to the motor and the second cable end is coupled to the substructure, and wherein the cable length is routed through each panel cable opening,
wherein, in an assembled configuration and upon application of the motor, each of the two or more panel assemblies is adapted to move between a first position and a second position,, and each panel moving in an independent direction with respect to each other panel.

11. The helmet of claim 10, further comprising a top panel assembly coupled to a top biasing assembly, wherein the one or more biasing elements of the top biasing assembly bias the panel of the top panel assembly in a first inward position, and whereupon placement of the helmet on the scalp moves the top panel assembly to a second position [outwardly relative to the outer shell].

12. An apparatus for positioning over and applying electromagnetic energy to a body organ, the apparatus comprising:
a substructure including two or more biasing assembly supports;
two or more biasing assemblies;
two or more panel assemblies, each being coupled to a corresponding biasing assembly; and
a plurality of illumination assemblies, at least one illumination assembly being coupled to each panel assembly, each illumination assembly including a light source,
wherein, in an assembled configuration and upon application of an external force, each of the two or more panel assemblies is adapted to move between a first position and a second position, each panel assembly moving in an independent direction with respect to each other panel assembly.

13. The apparatus of claim 12, wherein each illumination assembly further comprises an optical assembly comprising a base including a lens formed therein, and one or more light guides configured to direct light from the lens to the body organ.

14. The apparatus of claim 12, further comprising an actuator to apply the external force causing movement from the first position to the second position and/or to return from the second position to the first position.
